(19) United States
(12) Patent Application Publication
(10) Pub. No.: US 2003/0223904 A1
(43) Pub. Date: Dec. 4, 2003

(54) UV bulk mail irradiation system

(76) Inventor: Lucas Lakhdar Bacha, League City, TX (US)

Correspondence Address: Lucas L. Bacha, 711 West Bay Area Blvd., Suite 220, PO Box 57888, Webster, TX, 77598, US

(21) Appl. No.: 10/078,750

(22) Filed: Jun. 4, 2002

(51) Int. Cl. A61L 2/00; A62B 7/08; B01J 19/08

(52) U.S. Cl. 422/24; 422/1; 422/121; 422/186

(57) ABSTRACT

The UV Bulk Mail Irradiation System consists of a chamber for disinfecting mail that has become contaminated by pathogens and spores such as Bacillius anthracis (anthrax bacteria) and Bacillius magaterium sp. (anthrax spores). Contaminated mail is placed in the chamber. Consideration for the characteristics of the chamber in which the contaminated mail has been place, the location of the C-band ultraviolet lights, the duration of the sterilization process allow effective elimination of this biohazard from the contaminated mail.

### FIELD OF THE INVENTION

The present invention relates to the field of germicidal systems employing microorganism-destroying ultraviolet lights. In particular, the present invention relates to a system for producing enough watts of C-band UV energy in a sterilization chamber to disinfect mail contaminated with anthrax bacteria and anthrax spores in five (5) minutes of less.

### BACKGROUND OF THE INVENTION

After Sep. 11, 2001, it has become very apparent that our mail system has become a biohazard target for Terrorist across the globe. Numerous deaths have resulted from our citizens coming in contact with mail pieces infected with anthrax. These mail pieces were either filled with anthrax or they were laced with the biohazard due to cross-contamination from mail pieces travelling in our mail system.

The current methods that are being tried in an effort to sterilize the mail have been ineffective, unsafe, and damaging to the mail content. Using this invention is safe for the user and does not damage the content of the mail.

The C-band ultraviolet light used in this invention has always been known for its germicidal properties. Especially, the C-band ultraviolet light spectrum of 254 nm, which has been found to disrupt and mutilate pathogens DNA, leading to pathogens cell death.

### SUMMARY OF THE INVENTION

The present invention is a chamber for disinfecting mail that has become contaminated by pathogens and spores such as Bacillius anthracis (anthrax bacteria) and Bacillius magaterium sp. (anthrax spores). Ultraviolet lights of sufficient intensity are positioned within a disinfecting chamber where they irradiate mail pieces contaminated with pathogens and spores such as Bacillius anthracis (anthrax bacteria) and Bacillius magaterium sp. (anthrax spores). The sterilization chamber has an entrance and an exit. The contaminated mail pieces are brought in through the entrance, placed in the chamber, the doors are sealed, and he irradiation process begins. After the mail pieces are decontaminated, they are removed, a new batch of mail pieces takes their place, and the decontamination process starts all over again

### DESCRIPTION OF THE INVENTION

The present invention consists of a chamber 8 feet wide, 8 feet high, and 16 feet long with wood surfaces covered with aluminum paint or reflectant material. In the chamber, there are 18 fixtures, each containing 4 lamps putting out 13.8 watts of ultraviolet light energy each (see table 1 and table 2 below). These lamps are located one (1) meter (393 inches) in parallel about a center point were 90-98 number 10 mail envelopes (4⅛ in.×9½ in) will be placed for disinfection.

Chamber Specifications:

TABLE 1

| Chamber width | 8 | Ft | 243.84 | cm |
|---|---|---|---|---|
| Chamber height | 6 | Ft | 243.84 | cm |
| Chamber depth | 16 | Ft | 487.68 | cm |
| Fixture containing 4 lamps | 59.2 | Watts | | |
| Lamp UV power | 13.8 | Watts | | |
| # of lamps | 72 | | | |
| Lamp model | G36T6L | | | |
| Lamp arclength | 36 | In | 91.44 | cm |
| Lamp diameter | 15.6 | Mm | 1.58 | cm |
| Lamp radius | | | 0.79 | cm |
| Reflectivity | 65 | % | Aluminum | Paint |
| Lamp spacing | Axis-to-axis | | 2 | cm |
| Center point | x-coord | | 121.92 | cm |
| | y-coord | | 121.92 | cm |
| | z-coord | | 243.84 | cm |
| Front end of lamps | z-coord | | 198.12 | cm |
| Back end of lamps | z-coord | | 289.56 | cm |

TABLE 2
Lamp fixtures coordinates (4 lapms each fixture)

| Lamp fixture | x1 | y1 | z1 | x2 | y2 | z2 |
|---|---|---|---|---|---|---|
| # | cm | cm | cm | cm | cm | cm |
| 1 | 116.92 | 213.36 | 198.12 | 116.92 | 213.36 | 289.56 |
| 2 | 118.92 | 213.36 | 198.12 | 118.92 | 213.36 | 289.56 |
| 3 | 120.92 | 213.36 | 198.12 | 120.92 | 213.36 | 289.56 |
| 4 | 122.92 | 213.36 | 198.12 | 122.92 | 213.36 | 289.56 |
| 5 | 124.92 | 213.36 | 198.12 | 124.92 | 213.36 | 289.56 |
| 6 | 126.92 | 213.36 | 198.12 | 126.92 | 213.36 | 289.56 |
| 7 | 213.36 | 123.92 | 198.12 | 213.36 | 123.92 | 289.56 |
| 8 | 213.36 | 121.92 | 198.12 | 213.36 | 121.92 | 289.56 |
| 9 | 213.36 | 119.92 | 198.12 | 213.36 | 119.92 | 289.56 |
| 10 | 126.92 | 30.48 | 198.12 | 126.92 | 30.48 | 289.56 |
| 11 | 124.92 | 30.48 | 198.12 | 124.92 | 30.48 | 289.56 |
| 12 | 122.92 | 30.48 | 198.12 | 122.92 | 30.48 | 289.56 |
| 13 | 120.92 | 30.48 | 198.12 | 120.92 | 30.48 | 289.56 |
| 14 | 118.92 | 30.48 | 198.12 | 118.92 | 30.48 | 289.58 |
| 15 | 116.92 | 30.48 | 198.12 | 116.92 | 30.48 | 289.56 |
| 18 | 30.48 | 119.92 | 198.12 | 30.48 | 119.92 | 289.56 |
| 17 | 30.48 | 121.92 | 198.12 | 30.48 | 121.92 | 289.86 |
| 18 | 30.48 | 123.92 | 198.12 | 30.48 | 123.92 | 289.56 |

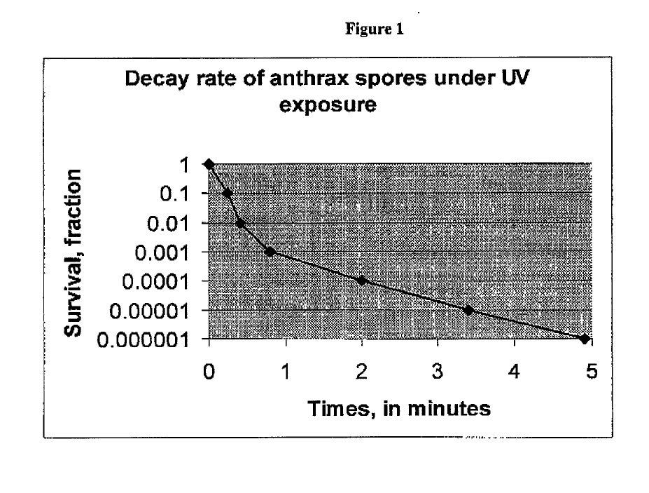

During the disinfection process, the average surface intensity will be at least 4690 μW/Cm² (see table 3). The time to sterilize mail contaminated with anthrax spores is estimated to be five (5) minutes or less (see FIG. 1).

TABLE 3
Average Surface Intensity - Horizontal Plane

| | | |
|---|---|---|
| Overhead lamps | 2304 | μW/Cm² |
| Side lamps | 158 | μW/Cm² |
| Total planar Intensity | 2462 | μW/Cm² |
| Reflective intensity at 65% | 2228 | μW/Cm² |
| Total planar intensity at surface 65% | 4690 | μW/Cm² |
| reflectivity | | |

What is claimed is:

1. A method for destroying pathogens and spores such as Bacillius anthracis (anthrax bacteria) and Bacillius magaterium sp. (anthrax spores) in a chamber having a set of wall and a ceiling panel comprising mounting ultraviolet lights, located in parallel about a center point were mail pieces will be placed for disinfection.

2. The method of claim 1, wherein the wall/ceiling/floor surfaces are painted with aluminum or paneled with reflectant material.

3. The method of claim 1, wherein 18 fixtures, each containing 4 lamps putting out 13.8 watts of C-band ultraviolet light energy each, located 1 meter (39.3 inches) in parallel about a center were mail will be placed for disinfection.

4. The method of claim 1, wherein during the disinfection process, the average surface intensity will be at least 4690 μW/Cm², sterilizing mail contaminated with anthrax spores within five (5) minutes or less.
